(19) United States
(12) Patent Application Publication
(10) Pub. No.: US 2014/0121531 A1
(43) Pub. Date: May 1, 2014

(54) DIAGNOSIS APPARATUS AND METHOD FOR ALZHEIMER'S DISEASE

(71) Applicant: Electronics and Telecommunications Research Institute, (US)

(72) Inventors: Kibong SONG, Daejeon (KR); Yong Ju YUN, Daejeon (KR); Kwan Su KIM, Seoul (KR); Yo Han CHOI, Daejeon (KR)

(73) Assignee: Electronics and Telecommunications Research Institute, Daejeon (KR)

(21) Appl. No.: 13/829,794

(22) Filed: Mar. 14, 2013

(30) Foreign Application Priority Data
Oct. 29, 2012 (KR) 10-2012-0120379

(51) Int. Cl. A61B 5/16; G01B 9/02

(52) U.S. Cl. CPC A61B 5/165; G01B 9/02091
USPC 600/476

(57) ABSTRACT

Provided are apparatus and method for diagnosing Alzheimer's disease. The diagnosis apparatus may include a rod type image probe obtaining signals on a state of an olfactory epithelium tissue region, the rod type image probe including an end portion configured to be inserted into a nose and be in contact with the olfactory epithelium tissue region, a delivering element, by which the signals may be transmitted from the olfactory epithelium tissue region, the delivering element being connected to other end portion of the rod type image probe, and a signal processing unit converting the signals transmitted from the delivering element into digital signals.

### CROSS-REFERENCE TO RELATED APPLICATIONS

This U.S. non-provisional patent application claims priority under 35 U.S.C. §119 to Korean Patent Application No. 10-2012-0120379, filed on Oct. 29, 2012, in the Korean Intellectual Property Office, the entire contents of which are hereby incorporated by reference.

### BACKGROUND OF THE INVENTION

Example embodiments of the inventive concept relate to diagnosis apparatus and method for Alzheimer's disease, and in particular, to apparatus and method for diagnosing Alzheimer's disease using optical coherence tomography.

Alzheimer' disease, also known in medical literature as Alzheimer disease, is the most common form of dementia (for example, accounting for about 50-80% of all dementia sufferers). In 2006, there were 26.6 million sufferers worldwide. Alzheimer's is predicted to affect 1 in 85 people globally by 2050.

Although there have been many researches about a method for curing Alzheimer disease, there is a paucity of research about a method for easily diagnosing Alzheimer disease.

Some common early symptoms of Alzheimer's disease include confusion, disturbances in short-term memory, problems with attention and spatial orientation, changes in personality, language difficulties and unexplained mood swings. For all that, since the cause for most Alzheimer's disease cases is still essentially unknown, it is hard to diagnose Alzheimer disease in the early stages. Pathologically, Alzheimer's disease is characterized by loss of neurons and synapses in the cerebral cortex and certain subcortical regions. In addition, Alzheimer's disease is also characterized by expansion of cerebral ventricles, loss of nerve cell, and neurofibrillary tangle and neuritic plaque of nerve fiber. Generally, a cognition ability test, a radiographic examination, pathological tests including blood and cerebrospinal fluid tests, olfactometry, eye examination, skin test and so forth may be used to diagnose Alzheimer disease.

One of main causes of Alzheimer disease may be a metabolic disorder and an accumulation of amyloid beta in brain. According to observations of an inner brain structure using a neuroimaging technique, called “proton magnetic resonance spectroscopy”, and a positron emission tomography (PET) technique, it turns out that Alzheimer disease is closely connected to not only accumulation of amyloid-beta in brain but also myoinositol/creatine and choline/creatine metabolite. However, in some cases, Alzheimer disease is diagnosed from a cognition ability test, regardless of a degree of accumulation of amyloid-beta in brain. This result shows that Alzheimer disease results from various biochemical and structural change of brain which begun several years ago.

Magnetic Resonance Imaging (MRI) and functional MRI (fMRI) may be used to analyze an encephalopathy. For example, an encephalopathy analyzer may include an image collecting part collecting MRI and fMRI related to an encephalopathy from each of the experimental and control groups, a volume analyzing part calculating a volume of cerebro-region from MRIs of the experimental and control groups and determining a first feature vector based on a difference in cerebro-region volume between the groups, an activation analyzing part calculating an activation of cerebro-region from fMRIs of the experimental and control groups and determining a second feature vector based on a difference in cerebro-region activation between the groups, and a feature extracting part extracting one of the first and second feature vectors as a feature vector to analyze the encephalopathy.

### SUMMARY

Example embodiments of the inventive concept provide an apparatus for diagnosing Alzheimer's disease using optical coherence tomography.

Other example embodiments of the inventive concept provide a method for diagnosing Alzheimer's disease using optical coherence tomography.

According to example embodiments of the inventive concepts, an Alzheimer's disease diagnosis apparatus may include a rod type image probe obtaining signals on a state of an olfactory epithelium tissue region, the rod type image probe including an end portion configured to be inserted into a nose and be in contact with the olfactory epithelium tissue region, a delivering element, by which the signals may be transmitted from the olfactory epithelium tissue region, the delivering element being connected to other end portion of the rod type image probe, and a signal processing unit converting the signals transmitted from the delivering element into digital signals.

In example embodiments, the rod type image probe may include an optical interferometer.

In example embodiments, the optical interferometer may include a light source, a beam splitter splitting a light emitted from the light source into a first split light and a second split light, an objective lens transmitting the first split light to the olfactory epithelium tissue region and transmitting a first reflection light reflected from the olfactory epithelium tissue region toward the beam splitter, a reference mirror receiving and reflecting the second split light to transmit a second reflection light toward the beam splitter, and a photodetector configured to detect a light including the first and second reflection lights combined at the beam splitter.

In example embodiments, the optical interferometer may further include a 3-axis actuator configured to be able to move the objective lens in three different directions.

In example embodiments, the objective lens may be configured to receive selectively a portion of the first reflection light scattered from a region around a focal depth of the objective lens.

In example embodiments, the light emitted from the light source may be a visible light or a near infrared light.

In example embodiments, the rod type image probe may include a buffer tip provided at the end portion thereof to be in close contact with the olfactory epithelium tissue region.

In example embodiments, the rod type image probe has a width of 20 mm or less.

In example embodiments, the delivering element may include an optical fiber.

In example embodiments, the apparatus may further include a computer configured to execute an image processing on the digital signals converted by the signal processing unit to display a result of the image processing as an image.

According to example embodiments of the inventive concepts, an Alzheimer's disease diagnosis method may include bring an end portion of a rod type image probe into contact with an olfactory epithelium tissue region through a nose to obtain signals on a state of the olfactory epithelium tissue region.

In example embodiments, the method may further include converting the signals obtained with the rod type image probe into digital signals.

In example embodiments, the method may further include performing an image processing on the digital signals and displaying a result of the image processing as an image.

In example embodiments, the rod type image probe may be configured to obtain the signals on the state of the olfactory epithelium tissue region using an optical interferometer.

In example embodiments, the optical interferometer may include a light source, a beam splitter splitting a light emitted from the light source into a first split light and a second split light, an objective lens transmitting the first split light to the olfactory epithelium tissue region and transmitting a first reflection light reflected from the olfactory epithelium tissue region toward the beam splitter, a reference mirror receiving and reflecting the second split light to transmit a second reflection light toward the beam splitter, and a photodetector configured to detect a light including the first and second reflection lights combined at the beam splitter.

In example embodiments, the optical interferometer may further include a 3-axis actuator configured to be able to move the objective lens in three different directions.

In example embodiments, the objective lens may be configured to receive selectively a portion of the first reflection light scattered from a region around a focal depth of the objective lens.

In example embodiments, the light emitted from the light source may be a visible light or a near infrared light.

In example embodiments, the rod type image probe may include a buffer tip provided at the end portion thereof to be in close contact with the olfactory epithelium tissue region.

In example embodiments, the rod type image probe has a width of 20 mm or less.

### BRIEF DESCRIPTION OF THE DRAWINGS

Example embodiments will be more clearly understood from the following brief description taken in conjunction with the accompanying drawings. The accompanying drawings represent non-limiting, example embodiments as described herein.

FIG. 1 is a schematic diagram illustrating an Alzheimer's disease diagnosis apparatus according to example embodiments of the inventive concept.

FIG. 2 is a schematic sectional view of a sample to be measured by the Alzheimer's disease diagnosis apparatus according to example embodiments of the inventive concept.

FIG. 3 is a schematic diagram illustrating a structure and operating principle of a component constituting the Alzheimer's disease diagnosis apparatus according to example embodiments of the inventive concept.

FIGS. 4A and 4B are images obtained by the Alzheimer's disease diagnosis apparatus according to example embodiments of the inventive concept.

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. For example, the relative thicknesses and positioning of molecules, layers, regions and/or structural elements may be reduced or exaggerated for clarity. The use of similar or identical reference numbers in the various drawings is intended to indicate the presence of a similar or identical element or feature.

### DETAILED DESCRIPTION

Example embodiments of the inventive concepts will now be described more fully with reference to the accompanying drawings, in which example embodiments are shown. Example embodiments of the inventive concepts may, however, be embodied in many different forms and should not be construed as being limited to the embodiments set forth herein; rather, these embodiments are provided so that this disclosure will be thorough and complete, and will fully convey the concept of example embodiments to those of ordinary skill in the art. In the drawings, the thicknesses of layers and regions are exaggerated for clarity. Like reference numerals in the drawings denote like elements, and thus their description will be omitted.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers indicate like elements throughout. As used herein the term “and/or” includes any and all combinations of one or more of the associated listed items. Other words used to describe the relationship between elements or layers should be interpreted in a like fashion (e.g., “between” versus “directly between,” “adjacent” versus “directly adjacent,” “on” versus “directly on”).

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises”, “comprising”, “includes” and/or “including,” if used herein, specify the presence of stated features, integers, steps, operations, elements and/or components, but do not preclude the presence or addition of one or more other features, integers, steps, operations, elements, components and/or groups thereof

Example embodiments of the inventive concepts are described herein with reference to cross-sectional illustrations those are schematic illustrations of idealized embodiments (and intermediate structures) of example embodiments. As such, variations from the shapes of the illustrations as a result, for example, of manufacturing techniques and/or tolerances, are to be expected. Thus, example embodiments of the inventive concepts should not be construed as limited to the particular shapes of regions illustrated herein but are to include deviations in shapes that result, for example, from manufacturing. For example, an implanted region illustrated as a rectangle may have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of example embodiments.

Unless otherwise defined, all terms (including technical and scientific terms) used herein have the same meaning as commonly understood by one of ordinary skill in the art to which example embodiments of the inventive concepts belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

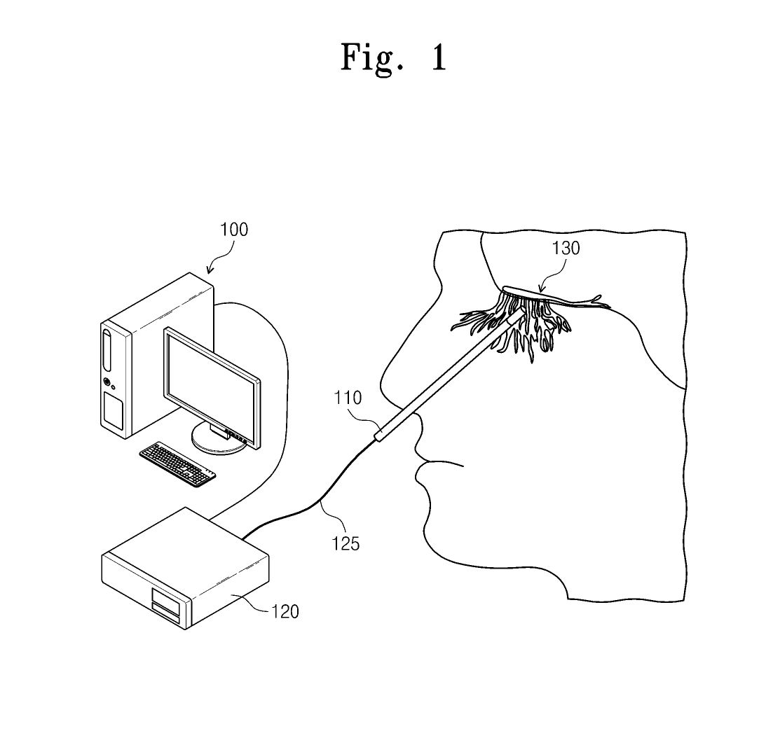

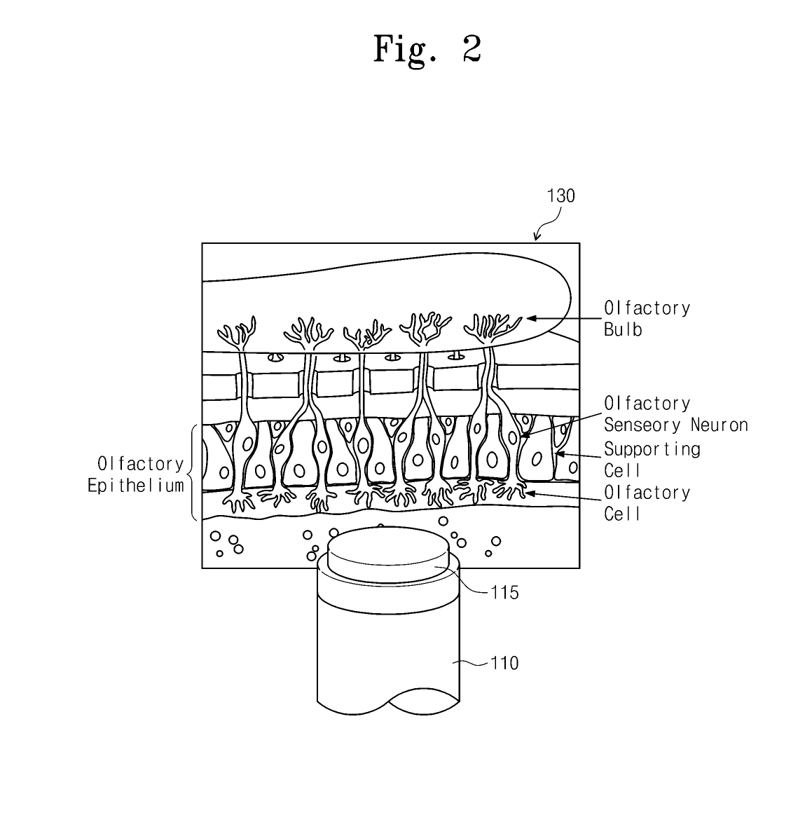

FIG. 1 is a schematic diagram illustrating an Alzheimer's disease diagnosis apparatus according to example embodiments of the inventive concept, and FIG. 2 is a schematic sectional view of a sample to be measured by the Alzheimer's disease diagnosis apparatus according to example embodiments of the inventive concept.

Referring to FIGS. 1 and 2, an Alzheimer's disease diagnosis apparatus may include a rod type image probe 110, a delivering element 125, a signal processing unit, 120 and a computer 100.

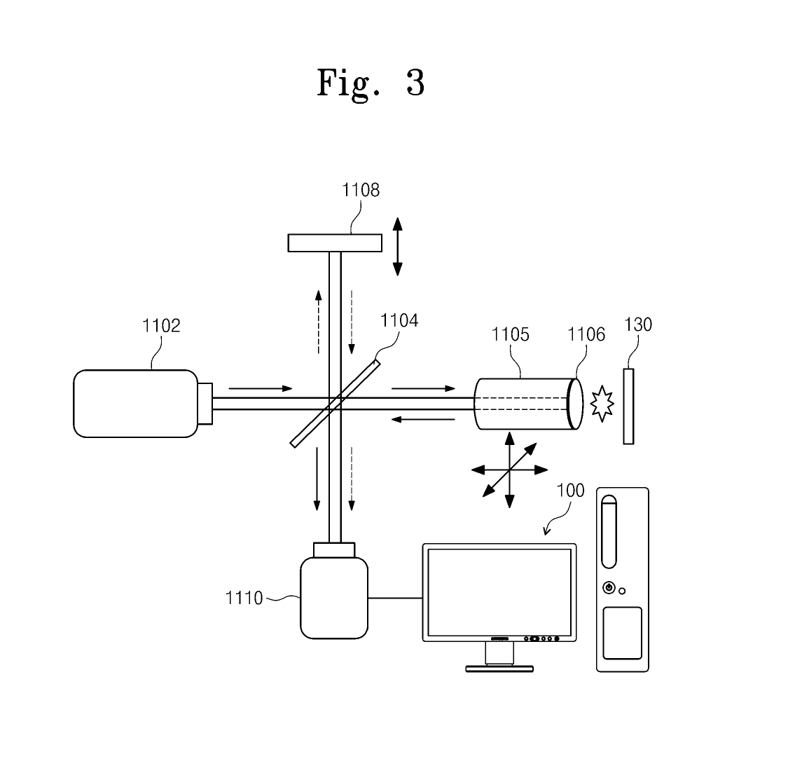

The rod type image probe 120 may be inserted into a nose to obtain signals showing a state of an olfactory epithelium tissue region 130. For this insertion of the rod type image probe 120 into the nose, the rod type image probe 120 may be configured to have a width of 20 mm or less. The rod type image probe 120 may have a circular transverse section, but example embodiments of the inventive concept may not be limited thereto. An end portion of the rod type image probe 110 may be in contact with the olfactory epithelium tissue region 130. The rod type image probe 110 may include an optical interferometer. The rod type image probe 110 may include a buffer tip 115 provided at the end portion thereof. Owing to the presence of the buffer tip 115, the rod type image probe 110 may be in close contact with the olfactory epithelium tissue region 130. For example, the buffer tip 115 may be connected at the end portion of the rod type image probe 110 using an elastic element, and thus, the end portion of the rod type image probe 110 may be controllably stuck to the olfactory epithelium tissue region 130. In addition, it is possible to control a focal depth of an objective lens (for example, see 1106 of FIG. 3), which may be provided in the optical interferometer of the rod type image probe 110. Accordingly, the use of the rod type image probe 110 enables to obtain information or signals on the olfactory epithelium tissue region 130 with ease.

The delivering element 125 may be connected to other end portion of the rod type image probe 110 to deliver the signals obtained from the olfactory epithelium tissue region 130 to, for example, the signal processing unit 120. The delivering element 125 may include an optical fiber, but example embodiments of the inventive concept may not be limited thereto. The use of the optical fiber enables to reduce a loss of the signals obtained from the olfactory epithelium tissue region 130.

The signal processing unit 120 may be configured to convert the signals, which are obtained from the olfactory epithelium tissue region 130 and delivered through the delivering element 125, into digital signals.

The computer 100 may execute an image processing on the digital signals converted by the signal processing unit 120 to display the result as an image. The computer 100 may be configured to contain software analyzing digital signals and displaying the analyzed result as an image.

The olfactory epithelium tissue region 130 may have an olfactory epithelium, which includes olfactory sensory neurons, supporting cells, and olfactory cilia, and an olfactory bulb connected thereto. Information or signals on the olfactory sensory neurons and the supporting cells of the olfactory epithelium of the olfactory epithelium tissue region 130 may be mainly obtained by using the rod type image probe 110.

FIG. 3 is a schematic diagram illustrating a structure and operating principle of a component constituting the Alzheimer's disease diagnosis apparatus according to example embodiments of the inventive concept.

Referring to FIG. 3, the rod type image probe (110 of FIG. 1) may include an optical interferometer. For example, the rod type image probe 110 may include an optical interferometer, in which low coherence interferometry or white-light interferometry is used. In example embodiments, the optical interferometer may be configured in the form of a Michelson interferometer, in which a light source 1102 having a very short coherence property is used.

The optical interferometer may include the light source 1102, a beam splitter 1104, a 3-axis actuator 1105, the objective lens 1106, a reference mirror 1108, and a photodetector 1110.

The light source 1102 may be configured to output a visible light having a wavelength of 380-780 nm or a near infrared light having a wavelength of 750-3,000 nm.

A light (depicted by a solid arrow toward the beam splitter 1104) emitted from the light source 1102 may be divided into a first split light (depicted by a solid arrow toward the objective lens 1106) and a second split light (depicted by a dotted arrow toward the reference mirror 1108) by the beam splitter 1104.

The objective lens 1106 may be configured to transmit the first split light onto the olfactory epithelium tissue region 130 and then receive and transmit a first reflection light (depicted by a solid arrow toward the beam splitter 1104), which is reflected by the olfactory epithelium tissue region 130, toward the beam splitter 1104.

The 3-axis actuator 1105 may be configured to be able to move the objective lens 1106 in three different directions. This is because the olfactory epithelium tissue region 130 is fixed with respect to a human body. In example embodiments, the objective lens 1106 may be jointed to an end portion of the 3-axis actuator 1105 using an adhesives or glue material. The 3-axis actuator 1105 may be provided in the form of tube. This enables to transmit the first split light toward the objective lens 1106 without interference. Accordingly, the objective lens 1106 may receive selectively a portion of the first reflection light scattered from a region around a focal depth of the objective lens 1106.

The reference mirror 1108 may be configured to receive and reflect the second split light to the beam splitter 1104. Accordingly, a second reflection light (depicted by a dotted arrow toward the beam splitter 1104) may propagate from the reference mirror 1108 toward the beam splitter 1104.

The first reflection light from the objective lens 1106 and the second reflection light from the reference mirror 1108 may be combined at the beam splitter 1104 and be detected by the photodetector 1110.

Due to the low coherence property of the light source 1102, the first and second reflection lights may interfere constructively or destructively with each other, when an optical path difference between the second reflection light from the reference mirror 1108 and the first reflection light from the olfactory epithelium tissue region 130 is shorter than a coherence distance of the light source 1102. Here, in the case where the reference mirror 1108 is moved parallel to a propagation direction of the second split light (e.g., in a manner of axial scanning or A-scanning) to increase an optical path of the second split light linearly, an interference fringe, which is produced by the first reflection light reflected from the olfactory epithelium tissue region 130, may vary according to a depth of the olfactory epithelium tissue region 130. Accordingly, if the reference mirror 1108 is moved with a constant speed, an interference fringe having a Doppler frequency may be detected by the photodetector 1110. Signals detected by the photodetector 1110 may be processed by an amplifier and a band-pass filter, and the use of the band-pass filter enables to remain signals having a component related to Doppler frequency. The signals having a component related to Doppler frequency may be processed by a modulator to obtain a size of a back-scatter light according to the depth of the olfactory epithelium tissue region 130. A desired two-dimensional section image may be obtained by repeating the afore-described process while moving a focus of the first split light along longitudinal and/or transverse directions using the 3-axis actuator 1105 attached with the objective lens 1106 (e.g., in a manner of longitudinal or/and lateral scanning or B-scanning).

Furthermore, the 3-axis actuator 1105 attached with the objective lens 1106 may be moved parallel to a propagation direction of the first split light (e.g., in a manner of axial scanning or C-scanning) to control a focal depth of the first reflection light. This enables to obtain a three-dimensional image of the olfactory epithelium tissue region 130.

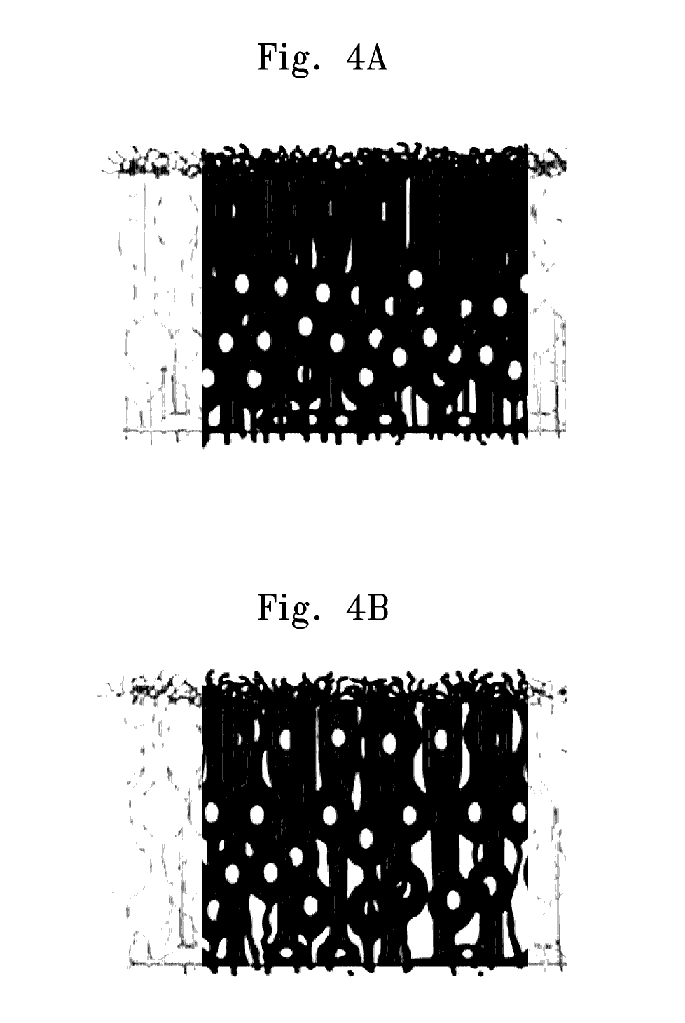

FIGS. 4A and 4B are images obtained by the Alzheimer's disease diagnosis apparatus according to example embodiments of the inventive concept.

FIG. 4A is an image of normal olfactory epithelium tissue region obtained from a person without Alzheimer's disease, and FIG. 4B is an image of abnormal olfactory epithelium tissue region obtained from a person with Alzheimer's disease.

As shown in FIG. 4A, in the case of normal olfactory epithelium tissue region, the olfactory sensory neurons were normally supported by the supporting cells. By contrast, as shown in FIG. 4B, in the case of abnormal olfactory epithelium tissue region, the olfactory sensory neurons were not normally supported by the supporting cells. This means that Alzheimer's disease can be easily diagnosed by observing an image showing whether the olfactory sensory neurons in the olfactory epithelium tissue region are properly supported by the supporting cells.

According to example embodiments of the inventive concept, an apparatus for diagnosing Alzheimer's disease may include a rod type image probe to obtain an image of an olfactory epithelium tissue region. The rod type image probe may be configured to be in contact with the olfactory epithelium tissue region and obtain information or signals on the olfactory epithelium tissue region. Accordingly, Alzheimer's disease can be easily diagnosed without a procedure to obtain blood and/or cerebrospinal fluid, an operation to obtain human tissue, or the use of expensive equipment to obtain brain tissue image. In other words, by using Alzheimer's disease diagnosis apparatus according to example embodiments of the inventive concept, it is possible to diagnose easily and cost-effectively Alzheimer's disease without complex process (such as procedure or operation) or expensive equipment.

According to example embodiments of the inventive concept, a method for diagnosing Alzheimer's disease may include obtaining an image of an olfactory epithelium tissue region using a rod type image probe. During diagnosis, the rod type image probe may be in contact with the olfactory epithelium tissue region to obtain information or signals on the olfactory epithelium tissue region. Accordingly, Alzheimer's disease can be easily diagnosed without a procedure to obtain blood and/or cerebrospinal fluid, an operation to obtain human tissue, or the use of expensive equipment to obtain brain tissue image. In other words, by using Alzheimer's disease diagnosis apparatus according to example embodiments of the inventive concept, it is possible to diagnose easily and cost-effectively Alzheimer's disease without complex process (such as procedure or operation) or expensive equipment.

Compared with the conventional diagnosis requiring a time-consuming pre-treatment using a procedure or operation and/or expensive large equipment, the apparatus and method according to example embodiments of the inventive concept may enable to diagnose Alzheimer's disease easily, quickly, and cost-effectively. Furthermore, the apparatus and method for diagnosing Alzheimer's disease according to example embodiments of the inventive concept can be used to improve understanding and monitoring of Alzheimer's disease and applied for various experimental treatments of Alzheimer's disease.

While example embodiments of the inventive concepts have been particularly shown and described, it will be understood by one of ordinary skill in the art that variations in form and detail may be made therein without departing from the spirit and scope of the attached claims.

What is claimed is:

1. An Alzheimer's disease diagnosis apparatus, comprising:
a rod type image probe obtaining signals on a state of an olfactory epithelium tissue region, the rod type image probe comprising an end portion configured to be inserted into a nose and be in contact with the olfactory epithelium tissue region;
a delivering element, by which the signals are transmitted from the olfactory epithelium tissue region, the delivering element being connected to other end portion of the rod type image probe; and
a signal processing unit converting the signals transmitted from the delivering element into digital signals.

2. The apparatus of claim 1, wherein the rod type image probe comprises an optical interferometer.

3. The apparatus of claim 2, wherein the optical interferometer comprises:
a light source;
a beam splitter splitting a light emitted from the light source into a first split light and a second split light;
an objective lens transmitting the first split light to the olfactory epithelium tissue region and transmitting a first reflection light reflected from the olfactory epithelium tissue region toward the beam splitter;
a reference mirror receiving and reflecting the second split light to transmit a second reflection light toward the beam splitter; and
a photodetector configured to detect a light including the first and second reflection lights combined at the beam splitter.

4. The apparatus of claim 3, wherein the optical interferometer further comprises a 3-axis actuator configured to be able to move the objective lens in three different directions.

5. The apparatus of claim 3, wherein the objective lens is configured to receive selectively a portion of the first reflection light scattered from a region around a focal depth of the objective lens.

6. The apparatus of claim 3, wherein the light emitted from the light source is a visible light or a near infrared light.

7. The apparatus of claim 1, wherein the rod type image probe comprises a buffer tip provided at the end portion thereof to be in close contact with the olfactory epithelium tissue region.

8. The apparatus of claim 1, wherein the rod type image probe has a width of 20 mm or less.

9. The apparatus of claim 1, wherein the delivering element comprises an optical fiber.

10. The apparatus of claim 1, further comprising a computer configured to execute an image processing on the digital signals converted by the signal processing unit to display a result of the image processing as an image.

11. An Alzheimer's disease diagnosis method, comprising bring an end portion of a rod type image probe into contact with an olfactory epithelium tissue region through a nose to obtain signals on a state of the olfactory epithelium tissue region.

12. The method of claim 11, further comprising converting the signals obtained with the rod type image probe into digital signals.

13. The method of claim 12, further comprising performing an image processing on the digital signals and displaying a result of the image processing as an image.

14. The method of claim 11, wherein the rod type image probe is configured to obtain the signals on the state of the olfactory epithelium tissue region using an optical interferometer.

15. The method of claim 11, wherein the optical interferometer comprises:
a light source;
a beam splitter splitting a light emitted from the light source into a first split light and a second split light;
an objective lens transmitting the first split light to the olfactory epithelium tissue region and transmitting a first reflection light reflected from the olfactory epithelium tissue region toward the beam splitter;
a reference mirror receiving and reflecting the second split light to transmit a second reflection light toward the beam splitter; and
a photodetector configured to detect a light including the first and second reflection lights combined at the beam splitter.

16. The method of claim 15, wherein the optical interferometer further comprises a 3-axis actuator configured to be able to move the objective lens in three different directions.

17. The method of claim 15, wherein the objective lens is configured to receive selectively a portion of the first reflection light scattered from a region around a focal depth of the objective lens.

18. The method of claim 15, wherein the light emitted from the light source is a visible light or a near infrared light.

19. The method of claim 11, wherein the rod type image probe comprises a buffer tip provided at the end portion thereof to be in close contact with the olfactory epithelium tissue region.

20. The method of claim 11, wherein the rod type image probe has a width of 20 mm or less.
